(19) United States
(12) Patent Application Publication
(10) Pub. No.: US 2019/0335817 A1
(43) Pub. Date: Nov. 7, 2019

(54) VAPORIZER DEVICE HAVING OUTPUT COMPONENT FOR COMMUNICATING AMOUNT OF GENERATED VAPORIZED SUBSTANCE

(71) Applicant: INDOSE INC., Woodland Hills, CA (US)

(72) Inventors: Daniel FREEMAN, Woodland Hills, CA (US); Ari FREEMAN, Woodland Hills, CA (US); Jacqueline FREEMAN, Woodland Hills, CA (US)

(73) Assignee: INDOSE INC, Weedland Hills, CA (US)

(21) Appl. No.: 16/516,683

(22) Filed: Jul. 19, 2019

Related U.S. Application Data
(63) Continuation of application No. 16/230,754, filed on Dec. 21, 2018.
Application No. 16/230,754 is a continuation of application No. 15/244,518, filed on Aug. 23, 2016.
(60) Provisional application No. 62/388,066, filed on Jan. 13, 2016.
(60) Provisional application No. 62/386,615, filed on Dec. 7, 2015.
(60) Provisional application No. 62/386,614, filed on Dec. 7, 2015.

(51) Int. Cl. A24F 47/00; A61M 15/00; A61M 15/06; G01N 30/00; G01N 21/85; F22B 1/28

(52) U.S. Cl. CPC A24F 47/008; A61M 15/0065; A61M 15/0086; A61M 2016/0021; G01N 30/00; G01N 21/85; F22B 1/284; A61M 15/06

(57) ABSTRACT

An inhalation device for inhaling a vaporized substance that includes metering capabilities to inform a user when a particular amount of substance has been consumed. The inhalation device can include a sensor that senses the vaporized substance and a processor that utilizes data from the sensor to meter the amount consumed. The inhalation device can also define a session, which is a time in which a user can consume a particular amount. During the session, a user can start and stop inhaling and resume inhaling. When the user stops inhaling the inhalation device will halt vapor production and will resume production when the user resumes inhaling.

### CROSS-REFERENCE TO RELATED APPLICATIONS

This is a continuation application of U.S. application Ser. No. 16/230,754, filed Dec. 21, 2018, which is a continuation of U.S. application Ser. No. 15/244,518, filed Aug. 23, 2016, which claims the benefit of the filing dates of U.S. Provisional Patent Application Nos. 62/386,614, 62/386,615, and 62/388,066, filed Dec. 7, 2015, Dec. 7, 2015, and Jan. 13, 2016, respectively. The above-name applications are incorporated by reference herein in their entireties.

### BACKGROUND

Inhaling devices such as vaporizers, vaporizing pens, and vaporizing machines are used to vaporize substances such as tobaccos, oils, liquids, medical drugs, and plant herbs. Once vaporized, these substances are then inhaled by consumers. Such inhaling devices have health benefits over traditional smoking methods. But inhaling the vapor can have negative effects on the body depending on the substance, such as nicotine. Inhaling devices have become more popular with consumers, but pose problems.

For example, while vaporizers can be safer than traditional smoking methods, it is difficult to meter the amount of vaporized substance that is being inhaled. So a user of an inhalation device that vaporizes nicotine may actually consume more nicotine than had the user smoked cigarettes or cigars.

There are multiple factors that affect the quantity of drug that is inhaled. These factors include the drug concentration of the vaporized substance, the amount of vapor inhaled, the duration of inhalation, variations between inhalation devices, and variation and inconsistency in the functionality of the device.

Another issue is that the inhaled substances may have different effects on different users depending on various factors. To optimize a user's experience, it is necessary to track the quantity inhaled taken over time and track the resulting effect it has on that user. This can be a tedious and demanding task. Typical users may not keep track of each dose and record the experience.

### SUMMARY

In one aspect, this disclosure describes an inhalation device for inhaling a vaporized substance that includes a channel through which the vaporized substance can flow, a light signal device, wherein the light signal device emits light; a sensor, wherein the sensor senses the light from the light signal device; and wherein the light signal device and the sensor are positioned in the channel such that the vaporized substance can flow past the sensor and the light signal device.

In another aspect, this disclosure also describes a processor, wherein said processor uses data from the sensor to meter the consumption of the vaporized substance. The inhalation device can also include a sensor, wherein the sensor acquires data relating to airflow in the device. The inhalation device can further include an indicator, wherein the indicator informs the user when a dose of the substance has been inhaled.

In another aspect, this disclosure describes an inhalation device for inhaling a vaporized substance including a processor; and a meter, wherein the meter includes an indicator; wherein the processor, using data from the timer, calculates the amount of the substance inhaled, and wherein the indicator informs the user of the amount that has been inhaled. The inhalation device can further include a mouthpiece, from which a user can inhale a vaporized substance; a reservoir, wherein the substance in unvaporized form is stored; and a heating element, wherein said heating element is used to heat the unvaporized substance.

The inhalation device can also have the capability of the meter indicating a progressive inhalation of the substance including a progressive inhalation of the substance in discrete quantities.

In another aspect, this disclosure describes an inhalation device including: a body, wherein the body includes: a mouthpiece, from which a user can inhale a vaporized substance; a reservoir, wherein the substance in unvaporized form is stored; a heating element, wherein said heating element is used to heat the unvaporized substance; and a processor, wherein the processor defines a session; wherein the device is configured such that the unvaporized substance from the reservoir is heated by the heating element to create a vaporized substance and said vaporized substance is inhaled by the user through the mouthpiece; and wherein the processor is configured to keep a session open, during which the processor is configured to stop the heating element when the user stops inhaling, and is configured to start the time and the heating element when the user resumes inhaling.

### DESCRIPTION OF THE DRAWINGS

FIG. 1A is a diagram of an inhalation device.

FIG. 1B is a diagram of a portion of an inhalation device.

FIG. 1C is another diagram of a portion of an inhalation device.

FIG. 2 is another diagram of an inhalation device.

FIG. 3 is another diagram of an inhalation device.

FIG. 4 is another diagram of an inhalation device.

FIG. 5 is another diagram of an inhalation device.

FIG. 6 graphically shows the relationship between optosensor output change and vapor intensity.

FIG. 7A schematically shows a vaporizer.

FIG. 7B schematically shows a dosage capsule.

FIG. 8A schematically shows a dosage vaporizer with a dosage control.

FIG. 8B schematically shows a dosage vaporizer having a touchscreen.

FIG. 9 schematically shows user devices connected to a cloud.

FIG. 10A schematically shows a dosage vaporizer having a button accepting an unlock code.

FIG. 10B schematically shows a dosage vaporizer having a biometric sensor.

FIG. 10C schematically shows a dosage vaporizer having a mechanical tension swipe.

FIGS. 11A and 11B schematically show a dosage vaporizer having buttons.

FIGS. 11C and 11D schematically show a dosage vaporizer having a touchscreen.

FIG. 11E schematically shows a dosage vaporizer having an inhale/exhale sensor for entering a pattern passcode.

FIG. 12A schematically shows a dosage vaporizer having a swipe sensor.

FIG. 12B schematically shows a button to be pressed to enter a Morse code type passcode.

### DETAILED DESCRIPTION

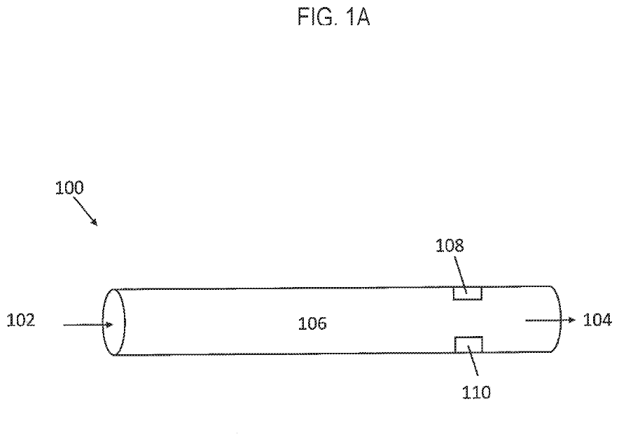

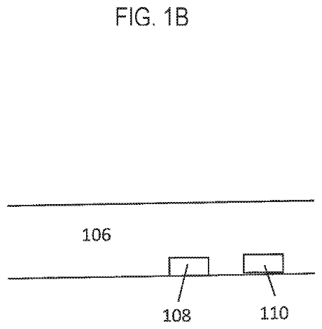

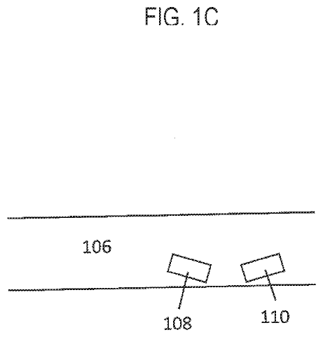

FIG. 1A illustrates an inhalation device 100, e.g., a vaporizer, for inhaling a vaporized substance. The inhalation device 100 includes a first opening 102 and a second opening 104. In between the two openings is a channel 106. When a user inhales using the inhalation device 100, air flows into the first opening 102 and in the inhalation device 100, vaporized substance is created by a heating element (not shown), and a mixture of air and vapor flows through the channel 106 to the second opening 104 and ultimately to the user.

The inhalation device 100 also includes a sensor 108 and a signal emitter 110. The sensor 108 and signal emitter 110 are positioned across from each other in the channel 106. The sensor 108 senses the vapor amount. For example, the sensor 108 can sense the concentration of vapor. The sensor 108 senses the intensity of the signal emitted by the signal emitter 110. If the sensor 108 senses a high signal output, this indicates that the amount of vapor is low, and the vapor/air mixture is dominated by air. Likewise, if the sensor 108 senses a low signal output, this indicates that the vapor/air mixture is dominated by vapor.

Data from the sensor 108 can assist the inhalation device 100 in providing information about vapor concentration to the user. For example, if the sensor senses a 5% drop in intensity from the signal emitter 110, that could correlate to a mixture of vapor/air that is 60% vapor.

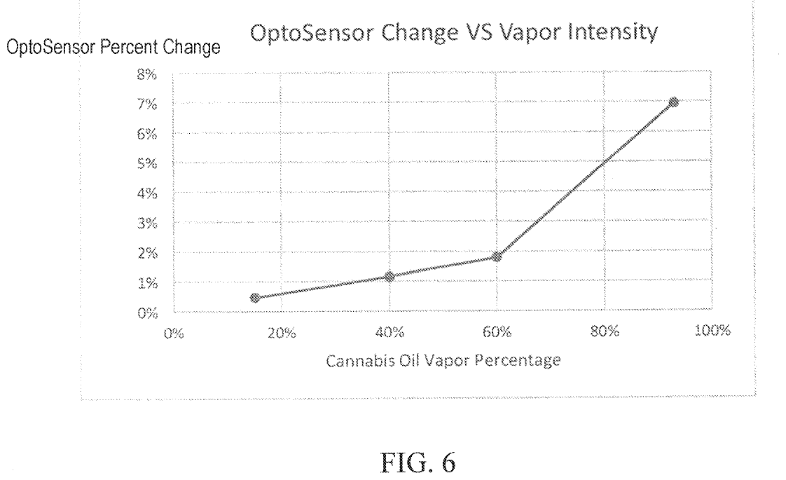

The chart of FIG. 6 graphs the value percent drop in an optocell (i.e., a device that senses the intensity of light) versus the percentage of cannabis oil vapor in a mixture of vapor and air.

Specifically, FIG. 6 shows the correlation between vapor concentration and the readings from an optocell. Knowing the relative concentration of the vapor can assist the inhalation device 100 in providing additional information to the user. For example, if a user inhales using the inhalation device 100 and the sensor 108 senses a high output, this may indicate that the concentration is less than expected. The inhalation device 100 could include an additional indicator to inform the user that the inhalation device 100 is not producing the expected amount of vapor. The sensor 108 can be any suitable sensor that senses light including without limitation, a photosensor, photodetector, optocell, optoresistor, optotransistor, optodiode, and/or solar cell. The signal emitter 110 can be any suitable device that produces light, such as an LED. The signal could also emit ultraviolet light. In other words, the signal emitter 110 can produce a wide range of wavelengths of light and the sensor 108 detects those wavelengths of light. The inhalation device 100 can optionally use filters in order to target a specific wavelength of light to optimally detect vapor intensity.

In FIG. 1A, the sensor 108 is positioned across from the signal emitter 110. The sensor 108 and the signal emitter 110 can also be positioned in alternative arrangements without departing from the scope of this disclosure. For example, in FIG. 1B the sensor 108 and the signal emitter 110 are positioned next to each other in the channel 106. In another embodiment, shown in FIG. 1C, the sensor 108 and the signal emitter 110 are positioned next to each other at an angle in the channel 106. The arrangements of the sensor 108 and the signal emitter 110 in FIGS. 1B and 1C use concepts of backscatter and fluorescence.

In backscatter, the vapor passing through the channel 106 can “reflect” light back from the perspective of the sensor 108. In this scenario, the vapor particle size would determine the “reflection” properties and angle of refection. In fluorescence, the light may get absorbed by the vapor particles and a new light may be generated. The new light would then be picked up by the sensor. The light and sensor may be set up facing the same direction (in parallel) towards the channel 106. Other alternative positions of sensor 108 and signal emitter 110 known to persons of ordinary skill in the art whereby the flow of vaporized substance affects the signal received by the sensor from the light produced by the light signal device is intended to fall within the scope of this disclosure. For example, the sensor 108 and the signal emitter 110 may be next to each other but one of the sensor 108 and the signal emitter 110 may also be positioned at an angle.

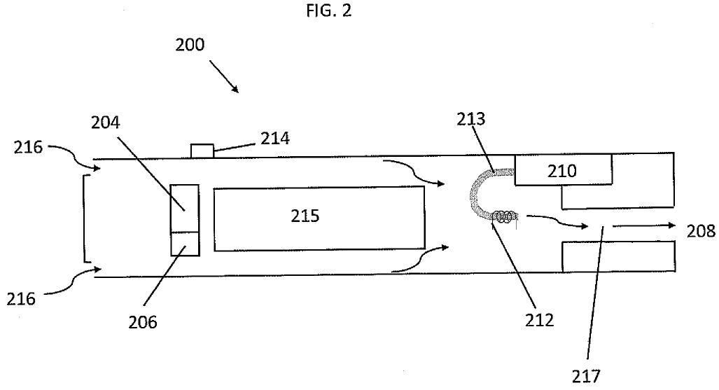

FIG. 2 shows an inhalation device 200. The inhalation device includes a processor 204 and a timer 206. In this embodiment, the inhalation device 200 includes an inlet 216, an outlet 208, a reservoir 210, a heating element 212, and a wick 213. The inhalation device 200 also includes an indicator 214 and a battery 215. The reservoir 210 stores the substance in unvaporized form, and the heating element 212 heats the unvaporized substance from the reservoir 210 via the wick 213 to create a vaporized substance, which is then inhaled by the user through the outlet 208. The inhalation device 200 also includes a channel 217 through which the vaporized substance produced by the heating element 212 and air will flow to the outlet 208 when a user inhales.

The inhalation device 200 uses the processor 204 and the timer 206 to provide metering information to the user. More specifically, the processor 204 controls the timer 206 such that when a user inhales using the inhalation device 200, the processor 204 will start the timer 206 as well as the heating element 212 to begin vaporizing the substance. After the timer 206 has reached a particular value, a particular amount of the vaporized element will have been produced, and the processor 204 will shut off the heating element 212. Alternatively, the processor 204 will not shut off the heating element 212, but rather will send a signal to the indicator 214 that the particular amount of the vaporized element has been consumed.

For example, if the heating element produces 1 mg/second, and the particular amount is 3 mg, the processor will turn on the heating element 212 when a user inhales, and the processor will turn off the heating element when the timer reaches 3 seconds. After the timer reaches 3 seconds, the processor will send a signal to the indicator 214, which will then indicate that the particular amount has been consumed. The indicator 214 can be an audio signal, visual signal, visual display, or a vibration. The indicator 214 could also be a transmitter that sends a signal to an external device such as a smart phone, tablet, or computer indicating that a particular amount has been consumed.

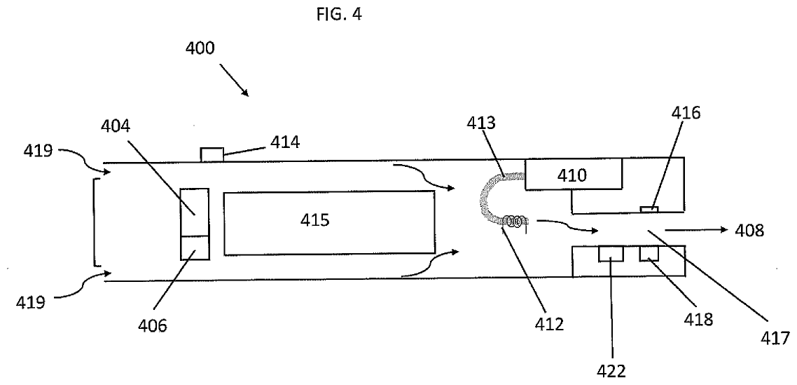

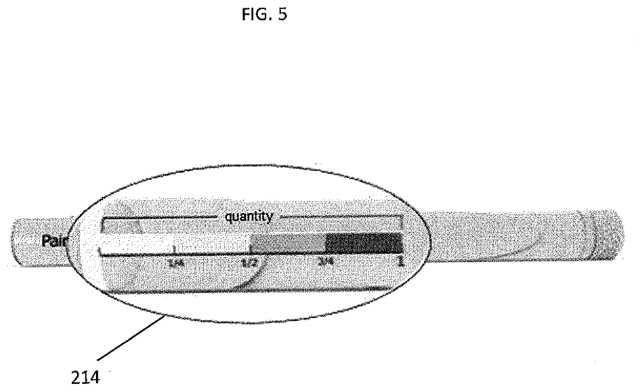

Alternatively, the indicator 214 could display what amount the user has consumed. As shown in FIG. 5, as a visual indicator to the user, the indicator 214 may include a progressive meter indicator. This could take the form of a sequence of lights, possibly LED lights, which indicate the progression of the amount consumed by the user. For example, there could be a sequence of four LED lights on the vaporizer indicating when a 25%, ½, 75% and full amount has been taken. When the full amount has been taken, the lights might be programmed to indicate to the user that the full amount has been reached by flashing. The progressive meter indicator could take other forms, like a mechanical indicator, a dial, a screen display, or a sound sequence. The progressive meter indicator may continue to meter and indicate the user consumption beyond one cycle. For example, after a full amount has been taken the indicator will turn all lights off and begin turning on each light again as the user consumes.

In the above example, in which a particular amount is set at 3 mg and the heating element 212 produces 1 mg/second of vapor, 3 mg will be delivered to a user who inhales for 3 seconds. In the event that the user cannot inhale long enough to consume a single dose in a single inhalation, the inhalation device 200 is configured to keep a session open, with a session being defined as a particular time within which a user can consume the particular amount. A session in this case could be set to 10 seconds. In this open session configuration, the inhalation device 200 can stop producing vapor when the user stops inhaling and start producing vapor when the user inhales again. When the sum of the user's inhalations amounts to consumption of 3 mg, the processor will send a signal to the indicator 214. Determining when the user stops inhaling can be achieved by using a pressure sensor. Where the pressure drops below a threshold, the heating element will stop. And when the pressure goes above the threshold, the heating element will resume. Alternatively, instead of time-based, a session can be vapor-based, where the inhalation device 200 keeps a session open until a certain quantity of vapor is produced.

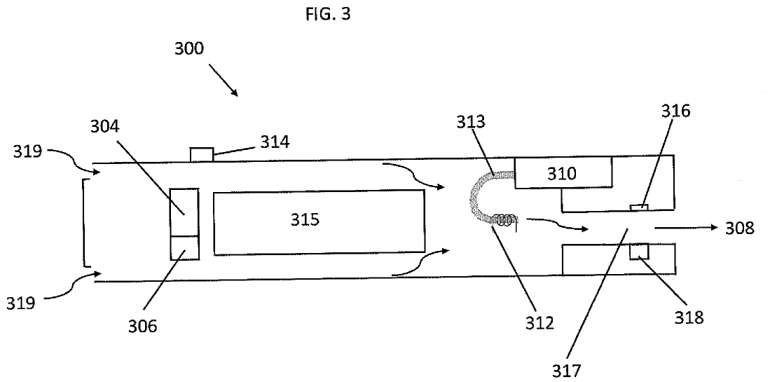

FIG. 3 shows an inhalation device 300 according to another embodiment. The inhalation device includes a processor 304 and a timer 306. In this embodiment, the inhalation device 300 includes an inlet 319, an outlet 308, a reservoir 310, a heating element 312, and a wick 313. The inhalation device 300 also includes an indicator 314 and a battery 315. The reservoir 310 stores the substance in unvaporized form, and the heating element 312 heats the unvaporized substance from the reservoir 310 via the wick 313 to create a vaporized substance, which is then inhaled by the user through the outlet 308. The inhalation device 300 also includes a channel 317 through which the vaporized substance produced by the heating element 312 and air will flow to the outlet 308 when a user inhales.

The inhalation device 300 further includes an indicator 314 that will indicate to the user when a particular amount of the vaporized substance has been consumed. The indicator 314 can be an audio signal, visual signal, visual display, or a vibration. The indicator 314 could also be a transmitter that sends a signal to an external device such as a smart phone, tablet, or computer indicating that a dose has been consumed. Alternatively, the indicator 314 could display what dose the user has consumed.

The inhalation device 300 can also include a sensor 316 and a signal emitter 318, such as an LED that produces a wide range of light wavelengths. The signal could also be one that produces ultraviolet light. The sensor 316 and signal emitter 318 are positioned across from each other in the channel 317. The sensor 316 senses the concentration of the vapor. For example, the sensor 316 can be an optical sensor that senses the intensity of the light produced by the signal emitter 318. If the sensor 316 senses a high output, this indicates that the vapor concentration is low, and the vapor/air mixture is mostly, if not all, air. If the sensor 316 senses a low output, this indicates that the vapor concentration is high. The processor 304 records information from the sensor 316. The sensor 316 can assist the inhalation device 100 in providing information about vapor concentration to the user. For example, if the sensor senses a 5% drop in intensity from the signal emitter 318, that could correlate to a mixture of vapor/air that is 60% vapor.

The processor 304 uses data from the sensor 316 to calculate when a particular amount of the vaporized substance has been produced. This is useful where the substance is viscous such as cannabis oil. In such viscous substances the amount of vapor produced for a given time can vary. In the embodiment of FIG. 3, when a user inhales using the inhalation device 300, the processor 304 will turn on the heating element 312. The sensor 316 will sense in real time (as a non-limiting example, every 0.1 seconds) the intensity of the light from the signal emitter 318. Using the data from the sensor 316, the processor 304 can determine when a particular amount has been produced.

For example, if a particular amount to be consumed is 3 mg and the heating element 312 vaporizes 1 mg per second, then theoretically the 3 mg should be produced in 3 seconds. In practice, however, it may take longer for the inhalation device 300 to vaporize 3 mg. This may be due to factors such as the time it takes the heating element 312 to heat up and the consistency of the drug released from the reservoir 310 to the wick 313. So for example, when a user begins to inhale, the first ten readings of the sensor 316 in the first second (e.g., one reading every 0.1 seconds) may indicate that the vapor produced over the first second is 50% of the expected production. This percentage can be thought of as a vapor factor. The processor 304 will take this vapor factor into account to determine when 3 mg is consumed by the user. In other words, the processor 304 will collect the data from the sensor 316 (e.g., every 0.1 seconds) on the vapor factor to determine when 3 mg has been consumed by the user. For a given time, the processor 304 will multiply the time (e.g., 0.1 seconds) by the vapor factor at that time, and will add each of these products to determine when a particular amount has been consumed. For example, if in the first second of inhalation, 50% of vapor is produced, and assuming 100% of vapor is produced after 1 second, the processor will able to determine that 3 mg has been consumed in 3.5 seconds.

In the above example, the processor 304 is capable of acquiring data from the sensor 316 and also included information on how much a particular amount of substance is expected to be produced per unit of time. The processor 304 can store additional vapor characteristics of the substance. For example, the processor 304 can store the time it takes for the heating element 312 to heat to the temperature at which it vaporizes the substance. The processor 304 can also store the heating and temperature variations during different inhalation profiles. For example, if a user inhales at a high rate, the air flowing through the inlet 319 and into the inhalation device 300 can cool the heating element 312. The processor 304 can store information on different rates of inhalation to adjust, for example, the temperature of the heating element 312. The processor 304 can also store information on the flow of drug from the reservoir 310 to the wick 313, the concentration of the substance within a given volume, and the vaporization rates of the substance at different temperatures of the heating element 312. The processor 304 as well as the processors discussed herein can be standard integrated circuit (IC) chips made by IC manufacturers such as Texas Instruments.

FIG. 4 illustrates another inhalation device 400 according to another embodiment of the disclosure. The inhalation device 400 includes a processor 404 and a timer 406. In this embodiment, the inhalation device 400 includes an inlet 419, an outlet 408, a reservoir 410, a heating element 412, and a wick 413. The inhalation device 400 further includes an indicator 414 for informing a user when a dose of the substance has been inhaled. The inhalation device 400 also includes a channel 417 through which air and the vaporized substance produced by the heating element 412 flow to the outlet 408 when a user inhales.

The inhalation device 400 also includes a sensor 416 and a signal emitter 418, such as an LED that produces a wide range of light wavelengths. The signal could also be one that produces ultraviolet light. The sensor 416 and signal emitter 418 are positioned across from each other in the channel 417. The sensor 416 senses the concentration of the vapor. For example, the sensor 416 can be an optical sensor that senses the intensity of the light produced by the signal emitter 418 at wavelengths that would include, but not be limited to, visible light and ultraviolet light.

The inhalation device 400 further includes a volume flow sensor 422. The sensor 422 can be any suitable airflow sensor including, but not limited to, any combination or stand-alone of the following: a pressure sensor, a propeller, a microphone or a piezoelectric sensor. The sensor 422 is used to measure the velocity at which the mixture of vapor and air flow through the channel 417. So for example, if the sensor 422 is a propeller, the propeller would be installed in the channel 417 and would spin according to velocity of the vapor/air mixture. The frequency of revolutions can be measured and used to calculate the velocity of the mixture. If the sensor is a microphone, the microphone can be setup in the channel 417 to listen to the noise of the vapor/air mixture passing through the channel. A correlation can be made between the sound intensity and/or frequency to the rate of flow of the mixture. Optionally, the sensor 422 can be placed between the inlet 419 and the processor 404 such that it detects the air flow rate going through the inhalation device 400 when a user inhales.

The sensor 422 can be used to adjust the intensity of the heating element 412. The temperature of the heating element can affect the amount of the substance that is vaporized. The sensor 422 is able to sense how intensely a user inhales (i.e., senses the volume per unit time of an inhalation). The processor 404 can acquire this data and adjust the intensity of the heating element by adjusting the voltage of the heating element.

The sensor 422 and the adjustment of the heating element 412 are useful in a non-limiting situation where the user desires to consume a dose more quickly. So for example, if the inhalation device 400 is set up so that the heating element produces 1 mg/second of vapor and a dose is 3 mg, a user that inhales at a high volume per unit time can consume the entire dose quicker than 3 seconds. In this scenario, the sensor 422 will be able to sense the higher velocity of the vapor/air mixture, and the processor can increase the intensity of the heating element such that it produces more vapor. The processor 404 can adjust the intensity of the heating element 412 in real time based on data from the sensor 422. So if a user does not inhale intensely, the sensor 422 will detect the decreased flow rate and the processor can then lower the intensity of the heating element 412.

In another embodiment, the inhalation devices described herein can be connected to a mobile device such as a smartphone or tablet and interfaced with a software application. The software application can record the doses that the user has inhaled and record the user's dosage experience. This information can be analyzed by the software to track and optimize the user's experience with the substance inhaled. To help improve analysis, the user could also enter personal information such as ailments, pains, weight and food intake. The information recorded can be used to accurately monitor a user's intake details and may be submitted to a doctor for review and/or improvement.

The application could also connect with other users via the internet. This could be used to share experiences, receive recommendations, and network with a community of users. The application may also be used as an ecommerce platform to purchase dosage capsules, or vaporizer equipment. The platform could offer specific substances based on a user's rated experience. Another enhanced use might be finding other users within geographic locations that may allow for social interactions and meetings. These enhanced services may be integrated with others over the internet.

The vaporizer device could also be locked by the user via the application. This could be used as a safety feature against undesired use (by children or others). There could be locking customizable lock setting to enhance safety or limit usage for those with low self-control.

As described above, vaporizers are used for an intake of the prescription and recreational drugs. However, it is difficult to meter the amount of drugs being inhaled. There are multiple factors that may affect the quantity of drug that is inhaled. These factors include the drug concentration of the vaporized substance, and the amount of vapor inhaled. Small changes in these factors can have big effects on the dosage inhaled.

Further, drugs may have different effects on different users depending on various factors. To optimize a user's experience and/or healing, tracking the dosage taken over time and tracking the resulting effect for a particular user may be needed.

Exemplary embodiments provide a simple yet effective solution and may be used together or separately.

As described above, a dosage indicator may be provided and may be a combination or stand-alone of a speaker, a vibrator, and lighting. The dosage indicator may be used to communicate with the user about the desired usage and the dosage of the vaporizer. For example, the dosage indicator might beep when the user has reached the desired dosage amount.

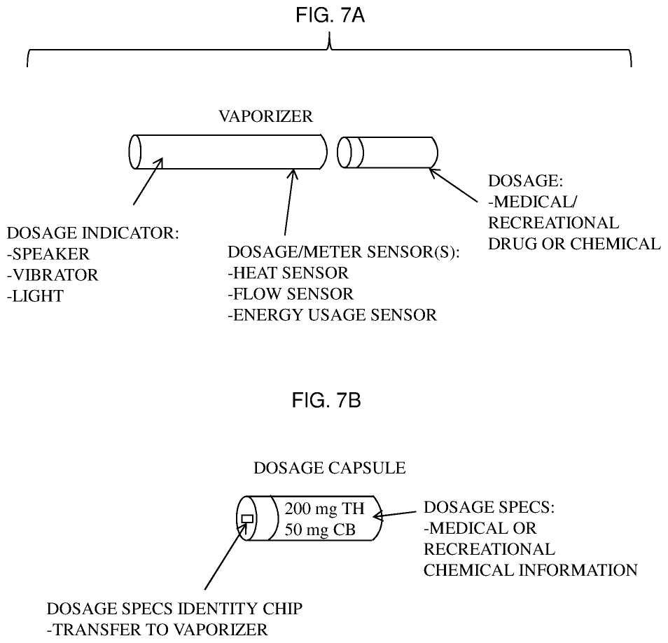

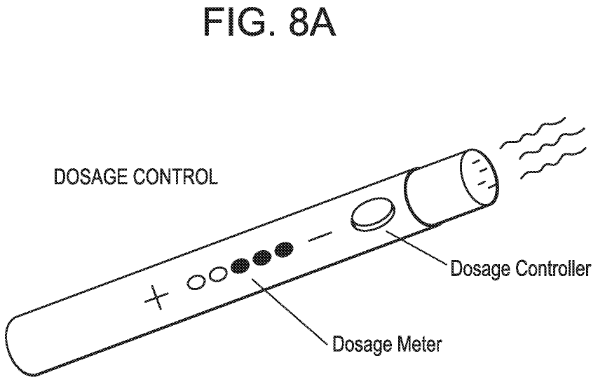

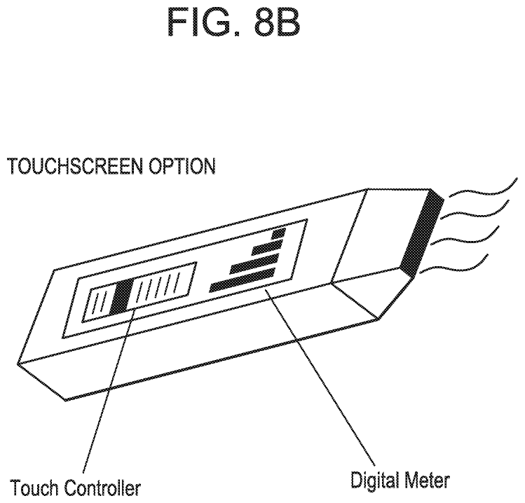

FIG. 7A schematically shows a vaporizer having a dosage indicator. FIG. 7B schematically shows a dosage capsule having dosage specifications. FIG. 8A schematically shows a dosage vaporizer with a dosage controller and a dosage meter. FIG. 8B schematically shows a dosage vaporizer having a touchscreen with a touchscreen controller and a digital meter.

Dosage meter(s) and/or sensor(s) may be provided and may be any combination or stand-alone of: time measurements, an air flow sensor, a mass flow sensor, a volume/measurement sensor for air, a volume/measurement sensor for the medication and/or drug, a heat sensor, current measurements, voltage measurements, a vapor analyzer, a vapor concentration sensor, or a vapor contents sensor. Other methods of detecting airflow may be performed by using pressure sensors, microphones as pressure sensors, microphones as sound sensors to detect air flow (for example, by detecting a whistle sound of the air). Other methods of measuring air flow directly or indirectly may also be used. By using some of the input information obtained by the above-mentioned means, the system may calculate and/or display the amount of drug intake. Alternatively, the system could be set to stop dispensing the drug once a certain dose is reached.

Exemplary embodiments may use many different brands or manufacturers of capsules such as generic capsules which are not made specifically for use with a specific device of an exemplary embodiment. In such cases, the dosing characteristics may vary from the capsules specifically designed. The studies may be performed with these capsules and their resulting characteristics could be used to fine tune the setting and the dosages. The information could be loaded into the inhalation device in a simple manner. When a capsule that is specifically made for a specific device of an exemplary embodiment is used, this will allow the user even more data and control on the dosage intake. Capsules specifically made may have identifying information that could be manually or automatically entered into the vaporizer. Knowing the identifying information from the capsule, the vaporizer may recognize the specifications about the drug and chemical compounds in the capsules. Knowing this information may allow the vaporizer to more accurately meter the dosage and improve performance. The optional dosage capsule could also be built into the vaporizer. One possible variation of the vaporizer may include a disposable or limited time use device.

Vaporizer may be connected to a mobile device such as a smartphone or tablet. A software application may provide the smartphone's interface with the vaporizer such that the users may monitor their usage through the software, save their dosage information, use information and/or rate their dosage experience. This information may be analyzed by the software to track and optimize the experience with the drug. To help improve analysis, the user could also enter personal information such as ailments, pains, weight, and food intake.

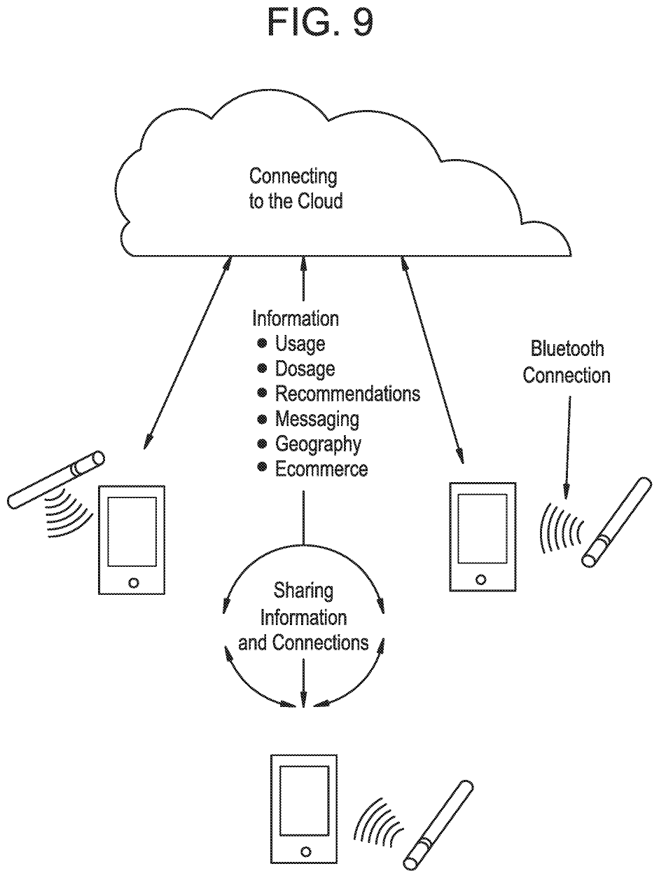

The system may monitor various drug ‘models’ and strains, and when each is used. The application may connect with other users via the Internet (see FIG. 9). This could be used to share experiences, receive recommendations, and network with a community of users. The application may also be used as an ecommerce platform to purchase dosage capsules, or vaporizer equipment. The platform could offer specific drugs based on the user's rated experience. Other services may be offered through the application, such as music tracks, software games, food offerings, and text messaging. Users could create their own experiences in the form of a ‘trip’. Example: take one dose of strain 1, on-screen mood lighting, play song 1, play song 2, two doses strain 2, video 1, game 1, 30 min free time, eat pizza, 1 dose strain 3, bath-time. These trips can be shared with other users. Another use might be finding other users within geographic locations that may allow for social interactions and meetings. These services may be integrated with other users over the Internet. The system can be used to accurately monitor a patient's intake details which may be submitted to a doctor for review and/or improvement. The vaporizer device could be locked via the application. This could be used as a safety feature against undesired use (by children or others). There could be locking via a customizable lock setting to enhance safety or limit usage for those with low self-control. The system could also help users understand their current state of ‘under the influence’ and warn the users against certain activities.

The application could also monitor and analyze other forms of drug intake not consumed via the vaporizer.

The vaporizers may be portable and battery operated. Many of the vaporizers are easily turned on and used. Some do not have an on/off button and are instantly turned on by a user inhaling from them. Unintended users may inhale the vapor without intending/knowing and the inhaling may be dangerous for some users, e.g., for a child. Further, the vaporizers are often meant for personal use only. Many times vaporizers contain product that is meant to be used by a specific person and not to be shared or used by others, as for example, when vaporizing prescription drug products. Also, parts within the vaporizers get extremely hot (approximately 400 degrees) and accidental turning on a vaporizer may have consequences.

According to embodiments, a vaporizer may have a lock/unlock and/or activate/deactivate feature. This feature can be mechanical, electrical, software, or a combination of these solutions.

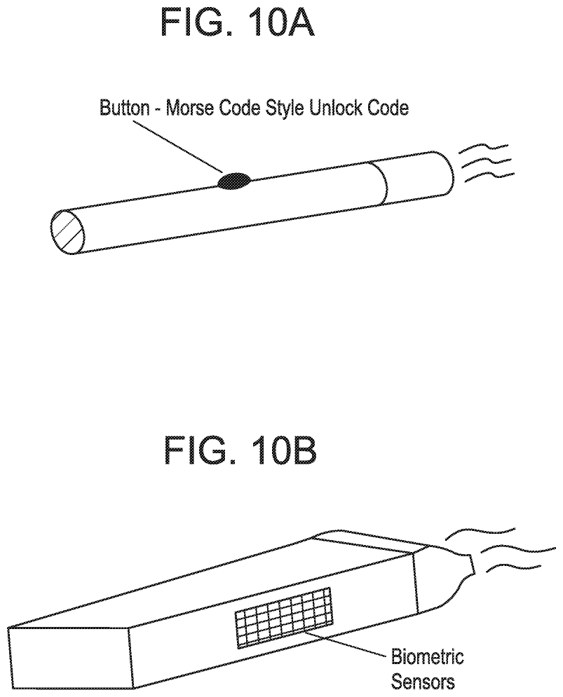

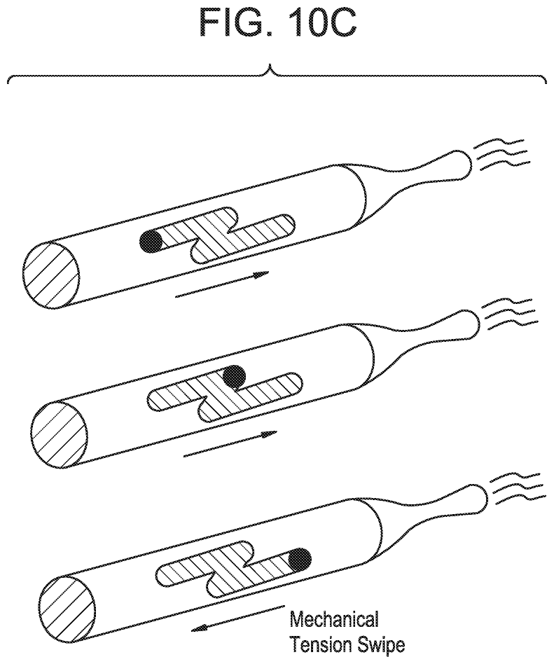

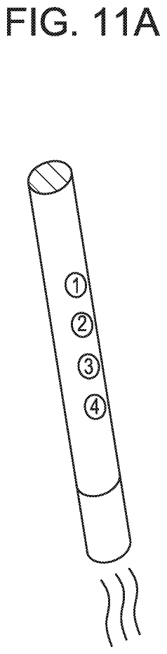

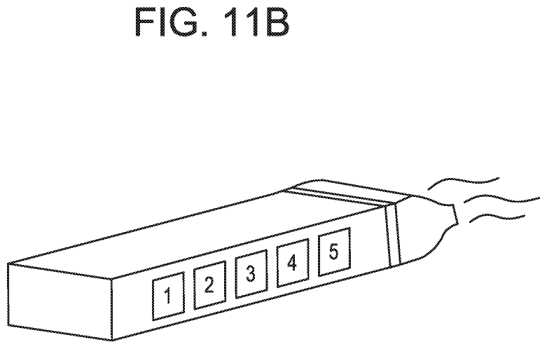

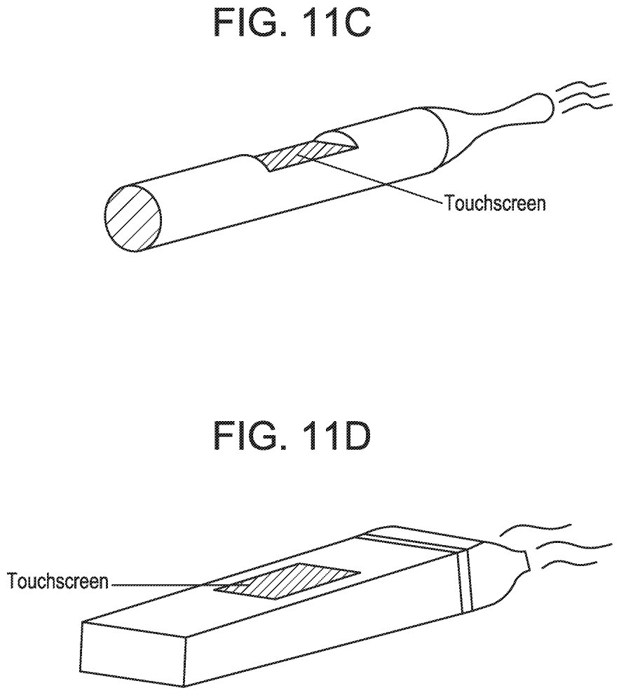

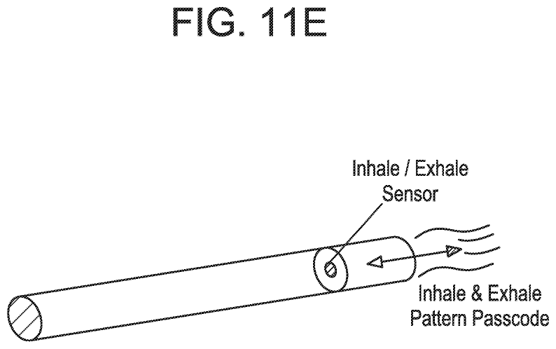

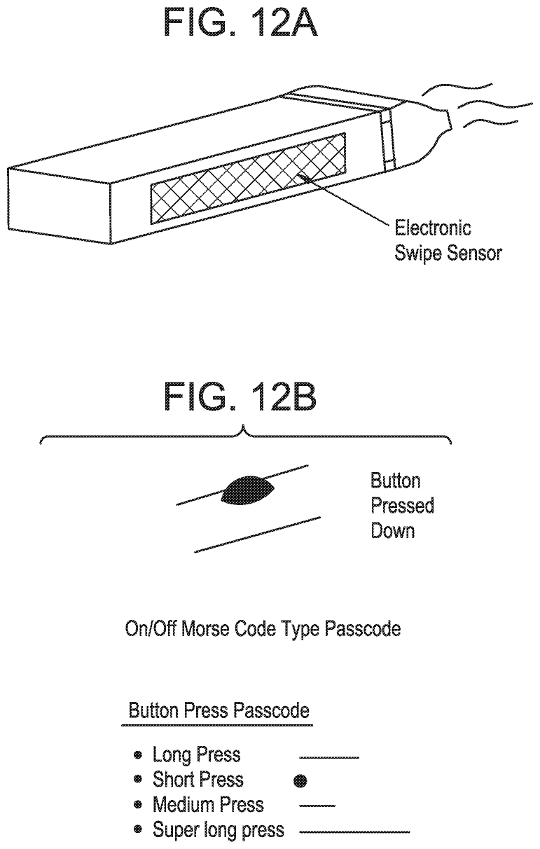

FIG. 10A schematically shows a dosage vaporizer having a button accepting an unlock code. FIG. 10B schematically shows a dosage vaporizer having a biometric sensor. FIG. 10C schematically shows a dosage vaporizer having a mechanical tension swipe. FIGS. 11A and 11B schematically show a dosage vaporizer having buttons. FIGS. 11C and 11D schematically show a dosage vaporizer having a touchscreen. FIG. 11E schematically shows a dosage vaporizer having an inhale/exhale sensor for entering a pattern passcode. FIG. 12A schematically shows a dosage vaporizer having a swipe sensor. FIG. 12B schematically shows a button to be pressed to enter a Morse code type passcode.

For example, as shown in FIGS. 10A and 12B, the device may include a button(s) that is pressed in a preprogrammed or customized pattern sequence which would unlock the vaporizing ability. This could work as a Morse code sequence acting as a passcode to enable the inhalation device. The code may include beeps of various lengths and pauses of various lengths, allowing complex codes with a single button.

For example, as shown in FIG. 10C, the device may include a mechanical locking device that would need a mechanical key or sequence of movements. The movements could be done with the user's hand, teeth, tongue, blowing, sucking and/or by shaking.

For example, the device may include a software key, passcode, or biometric reading to enable the device (see FIG. 10B). Further options may include a mechanical resistance feature that would be difficult for a child's dexterity to enable, such as a sliding bar. Other possible options may include requiring the user to successfully complete a specific swipe pattern with a finger on the device (see FIG. 12A). Other possible options may include biometric sensors that can be programmed to recognize specific users. Other possible iterations could include a multiple buttons with or without identifying numbers on them (see FIGS. 11A and 11B). Users may use the buttons to enter a passcode made up of a sequence of button presses, a sequence of numbers, a sequence of letters, or a mix of letters/numbers.

For example, a passcode may be required that is entered by inhaling or exhaling on the vaporizer, as shown in FIG. 11E. The inhales and/or exhales may act in place of the button presses and may allow the user to enter a Morse code style passcode.

For example, as shown in FIGS. 11C and 11D, the device may include a touch screen. Users may enter into the touchscreen a passcode to unlock or activate the vaporizer. Passcodes could be defined by the user and/or come preprogrammed by the factory. The software may be provided to allow the users to create multiple passcodes which may have multiple different restrictions or parameters such as user identification information, limit usage, limit drug dosage, auto lock settings. User specific information may be stored in a data log locally on the device or on other connected devices such as smartphones, smart watches, etc. The vaporizer could connect to other devices, such as smartphones, smartwatches, computers, smart home hubs, via Wi-Fi, Bluetooth, or a cable connection.

As described above, the device may include a dosage indicator as any combination or stand-alone of a speaker, a vibrator, and lighting. The indicators may communicate a partial dosage or multiple doses.

The examples of sensors that may be used for dosage metering are:
Air pressure sensors setup to measure the pressure at various positions in the inhale tube. These measurements can be compared to each other and based on the distance between the sensors and diameter of tube, the airflow rate and/or volume may be determined.
A propeller may be set in the tube that would spin according to air speed. The frequency of revolutions could be measured and used to calculate air speed.
A microphone may be set inside the inhale tube to listen to the white noise of the air passing through. A correlation may be made between the sound intensity and/or frequency to the airflow rate.

The above information may be combined with known vaporization characteristics of the vape, vapor characteristics and/or other measured data (e.g., time) and then a determination may be made about the drug dosage of the inhale. Using some of the above inputs, the system could calculate and/or display the amount of drug intake.

Alternatively, the system could be set to stop dispensing the drug once a certain dose is reached. The vaporizer unit may be designed so that the airflow rate is known by design. For example, the design may limit the flow rate by restricting the airflow to a known airflow rate, perhaps by directing the flow through a narrow channel. In such a case, the airflow rate would be known and direct airflow rate measurements might not be needed. Rather, the known airflow rate could be combined with other factors, such as duration (time) of inhale and other vaporization characteristics, to determine the quantity of drug consumed.

The measured information may be combined with specific characteristics of the vaporizer unit to determine consumption information. For example, a flow rate of 20 cm³/second combined with an inhale duration of 3 seconds will result in a 60 cm³ volume intake. This information may be combined with a drug-vapor-density factor (e.g., 1 mg drug/100 cm³) to determine the quantity of drug consumed (in this case 0.6 mg of drug). Further accuracy may be achieved by incorporating information regarding the vaporization element, such as current, voltage, startup time delays and so on.

Other methods of metering could include metering of the un-vaporized drug and metering the delivery from the cartridge to the heating chamber.

Examples of such embodiments may include:
A metered valve to monitor the drug delivered from the storage to the heating area.
An optical sensor configured to measure the remaining drug in the chamber.
Weight measurements to compare pre- and post-delivery weights of the drug.

Information may also be used to control the operation of the vapor generating element. For example, it may be desirable to generate more or less vapor depending on the air flow rate. This may allow for better control of the amount of drug in the dosage. Such means may entail adjusting the vaporization rate. Such adjustments may be accomplished by current and/or temperature variations. Another means may be switching the vaporization element on and off in a manner that results in the control of the vaporization rate.

The vaporizer characteristics may also play a role in the determination of dosage amount and concentration, as for example:
startup delay in vaporization means (such as a delay caused by a heating coil reaching the desired temperature),
vapor concentrations created at various coil temperatures,
vapor concentrations created at various voltage and current,
vapor concentrations created at various airflows,
drug concentrations with vapor,
flow characteristics of the air within the vaporizer,
frequency of the vaporizer use (which may affect a vaporizer performance),
time based variations, and
angle of vaporizer (which may cause performance variations).

Determination of the above factors may be calculated or tested. The results would be integrated into an algorithm. The algorithm would appropriately consider the various factors and make a determination on dosage. Based on the recommendations/signals of the algorithm, the user would be informed of the dosage information. For example, the usage information may be saved on the vaporizer or through the vaporizer on a smartphone, through the vaporizer and smartphone onto the cloud (see FIG. 9), or any combination of these options.

To prevent overdosing, an exemplary embodiment may set limits on the amount of drug inhaled in any defined period. To remind users to take their dosage, an exemplary embodiment may provide a reminder notification. The notification could take several forms, such as, audio, vibration, lights, and could also include connecting to a smartphone and sending a message to a user or a caretaker.

As described above, the device may be wirelessly connected to a smartphone (or other device) and software application may be provided to interface with the vaporizer. The application may be used as an ecommerce platform to purchase dosage capsules, or the vaporizer equipment. The platform may offer specific drugs based on user's rated experience. The application may automatically place an ecommerce order for additional vaporizing materials, such as a prefilled capsule or a replacement vaporizer. This could be triggered when the vaporizer senses it is getting low on extract oil, substrate, batteries, tobacco, drug, wax, and/or vaporizing liquid.

While embodiments have been described herein with a wick and heating element, other suitable methods of vaporizing a substance could be utilized without departing from the scope of this disclosure. For example, the substance to be vaporized could be placed in a chamber or oven. The oven can be a small cup made of metal, where a user could place the substance. The oven would then heat up and vaporize the substance. Any vapor produced can exit the oven and flow to the user when the user inhales.

While embodiments have been illustrated and described herein, it is appreciated that various substitutions and changes in the described embodiments may be made by those skilled in the art without departing from the spirit of this disclosure. The embodiments described herein are for illustration and not intended to limit the scope of this disclosure.

1. A device, comprising:
a memory configured to store one or more instructions; and
one or more processors configured to execute the one or more instructions to:
determine an amount of a vaporized substance generated by the device in a time period, and
provide, via an output component of the device, information related to the amount of the vaporized substance generated by the device.

2. The device of claim 1, further comprising a vaporization element configured to generate the vaporized substance,
wherein the one or more processors is further configured to execute the one or more instructions to:
determine a dosage of a medicant present in the vaporized substance generated by the vaporization element in the time period, and
control the vaporization element to generate the vaporized substance based on the determined dosage.

3. The device of claim 2, further comprising a sensor configured to detect a vapor information associated with the amount of the vaporized substance, the sensor comprising at least one from among an optical sensor, an air flow sensor, a mass flow sensor, an air volume measurement sensor, and a substance volume measurement sensor, and
the one or more processors is further configured to determine the dosage of the medicant based on the vapor information.

4. The device of claim 3, wherein the memory is further configured to store a target dosage of a user, and
the one or more processors is further configured to perform a comparison of the target dosage and the determined dosage, and control the output component to output a notification related to a result of the comparison.

5. The device of claim 1, wherein the output component comprises at least one from among a speaker, a vibrator, and a light.

6. The device of claim 1, further comprising:
a housing which houses a vaporization element and is configured to engage with a capsule containing a chemical compound,
wherein the one or more processors is further configured to control the vaporization element to generate the vaporized substance based on information about the chemical compound.

7. The device of claim 6, further comprising a dosage controller configured to receive a user input,
wherein the information about the chemical compound is input by a user via the dosage controller.

8. The device of claim 6, wherein the one or more processors is further configured to obtain the information about the chemical compound by reading a chip embedded in the capsule.

9. A method for controlling a vaporizer configured to generate a vaporized substance, the method comprising:
determining an amount of the vaporized substance generated by the vaporizer in a time period; and
providing, via an output component of the vaporizer, information related to the amount of the vaporized substance generated by the vaporizer.

10. The method of claim 9, further comprising:
sensing, by a sensor of the vaporizer, a vapor information associated with the amount of the vaporized substance;
determining a dosage of a medicant associated with the vaporized substance in the time period, based on the vapor information; and
controlling a vaporization element of the vaporizer to generate the vaporized substance based on the determined dosage.

11. The method of claim 10, wherein the sensor includes at least one from among an optical sensor, an air flow sensor, a mass flow sensor, an air volume measurement sensor, and a substance volume measurement sensor.

12. The method of claim 10, further comprising:
storing, in a memory of the vaporizer, a target dosage of a user;
performing a comparison of the target dosage and the determined dosage; and
controlling the output component to output a notification related to a result of the comparison.

13. The method of claim 9, wherein the output component includes at least one from among a speaker, a vibrator, and a light.

14. The method of claim 9, further comprising:
storing, in a memory of the vaporizer, a target dosage of a user; and
controlling a vaporization element of the vaporizer to generate the target dosage based on information about a chemical compound contained in a capsule engaged with the vaporizer.

15. The method of claim 14, wherein the controlling further comprises obtaining the information about the chemical compound by reading a chip embedded in the capsule.

16. A computer-readable storage medium storing instructions thereon which, when executed by one or more processors, cause the one or more processors to execute a method for controlling a vaporizer to generate a vaporized substance, the method including:
determining an amount of the vaporized substance generated by the vaporizer in a time period; and
providing, via an output component of the vaporizer, information related to the amount of the vaporized substance generated by the vaporizer.

17. The computer-readable storage medium of claim 16, wherein the method further includes:
controlling a sensor of the vaporizer to detect a vapor information associated with the amount of the vaporized substance;
determining a dosage of a medicant associated with the vaporized substance in the time period, based on the vapor information; and
controlling a vaporization element of the vaporizer to generate the vaporized substance based on the determined dosage.

18. The computer-readable storage medium of claim 17, wherein the method further includes:
storing, in a memory of the vaporizer, a target dosage of a user;
performing a comparison of the target dosage and the determined dosage; and
controlling the output component to output a notification related to a result of the comparison.

19. The computer-readable storage medium of claim 16, wherein the method further includes:
storing, in a memory of the vaporizer, a target dosage of a user; and
controlling a vaporization element of the vaporizer to generate the target dosage based on information about a chemical compound contained in a capsule engaged with the vaporizer.
